(19) United States
(12) Patent Application Publication
(10) Pub. No.: US 2004/0073258 A1
(43) Pub. Date: Apr. 15, 2004

(54) Body tissue and skin treatment method using pulsing heating pad and topical cream

(76) Inventors: W. Edward Church, Tampa, FL (US); Randall Krafft, Tampa, FL (US); Peter T. Pugliese, Bernville, PA (US); Peter M. Pugliese, Bethel, PA (US)

Correspondence Address: PEDERSEN & COMPANY, PLLC, P.O. BOX 2666, BOISE, ID, 83701, US

(21) Appl. No.: 10/454,744

(22) Filed: Jun. 3, 2003

Related U.S. Application Data
(63) Continuation-in-part of application No. 09/228,771, filed on Jan. 6, 1999, now Pat. No. 6,572,871.

(51) Int. Cl. A61N 1/00

(52) U.S. Cl. 607/1

(57) ABSTRACT

A body tissue treatment system comprising a heating device and a topical cream comprises controlled, non-burning heat exhibiting greater temperature fluctuation than a conventional heating device. At high temperature settings, the heating elements fluctuate widely between a relatively high maximum and low minimum to produce periods of intense heat input into the skin followed by sufficient cooling to prevent burning of the skin. Such application of intense heat to the skin, for short periods in which temperatures reach their peaks, relaxes musculature surrounding the injured area and significantly improves blood flow, skin permeability, and sweat gland openness in the area, which allows the topical cream to more readily penetrate skin and/or injured body tissues. While the skin temperature rises and falls to some extent in response to the heating device, the intense heating periods are believed to cause such high blood flow and consequent heat transfer away from the skin surface that the skin temperature does not rise to the extent of being damaged or burned.

This application is a continuation-in-part application of, and claims priority from, U.S. patent application Ser. No. 09/228,771, filed on Jan. 6, 1999, entitled “Pain Treatment Method and Apparatus Using Heating Wrap and Analgesic Cream,” issuing on Jun. 3, 2003 as U.S. Pat. No. 6,572,871, the disclosure of which is incorporated herein by this reference.

### BACKGROUND OF THE INVENTION

1. Field of the Invention

This invention relates, in general, to apparatus and methods for pain treatment or other treatment of body tissue by applying topical creams, including lotions, ointments, medicines or drugs, to the skin. More specifically, this invention relates to methods and apparatus for pulsed heating of the skin area on which has been placed a topical cream, which allows the topical cream to penetrate further into the skin for increased effectiveness of the active components of the cream. In a preferred embodiment, methods and apparatus are provided for reduction or elimination of pain using a heating pad in combination with an analgesic cream. In another embodiment, the methods and apparatus are used with moisturizing topical creams for general skin and/or face care.

2. Related Art

A variety of commercially available topical analgesic ointments and creams exist for the treatment of some forms of pain. The use of heat to reduce some pain symptoms is also known. However, prior to the development of a heat-compatible analgesic cream by the present inventors, heating devices and analgesic creams were not recommended for use in combination.

Studies have shown that the application of heat to the skin may have the capacity to relax muscles, increase blood flow at the skin surface and increase skin permeability. These factors promote penetration of topical creams through the skin into body tissues beneath the skin. Consequently, some topical pain treatment creams utilize heat generated by capsaicin-initiated exothermic reactions to increase penetration of the cream into the skin. However, no existing pain treatment method permits the application of intense heat to the skin surface, for the purpose of improving the penetration and effectiveness of topical analgesics, in a manner that is both safe and easy to implement.

It is an object of the present invention to address the need for improved body tissue treatment and skin care, for example, including pain treatment of injured tissue and joints and moisturizing treatment of the face and other skin areas. It is further an object of the embodiments of the present invention to provide a pain treatment system incorporating a novel, heat-compatible topical cream and pulsed heating methods and apparatus for enhancing penetration into the skin and body tissues.

### SUMMARY OF THE INVENTION

According to the objects of the invention, the present invention may comprise a treatment method including a heating apparatus combined with a treatment substance applied to the skin. Preferably, the apparatus and substance are adapted for pain treatment, utilizing an analgesic cream, or for general skin care, utilizing a moisturizing topical cream.

In the preferred embodiment, thermostatically-controlled heating elements within a heating pad alternate periodically, or pulse, between particular minimum and maximum temperatures that represent more substantial temperature variation than may be exhibited by conventional heating devices. The heating elements produce heat which may be safely applied to the skin surface over an injured area and/or a topical cream. The application of heat to the skin surface relaxes muscles surrounding the injured area, increases local blood flow to the area and increases skin permeability, which is particularly effective for pain treatment by topical analgesic cream.

The thermostatically-controlled heating pad of the preferred embodiment is specially-adapted to have at least one control setting wherein the temperature fluctuates in a wave pattern having a substantial difference between the peak temperature(s) and the trough temperature(s) of the surface of the heating pad. Preferably, this difference is at least 5 degrees F. but, more preferably, at least 10 degrees F., and typically in the range of 10-20 degrees F. Peak temperatures in the range of 135-145 F. measured on the surface of the heating pad may be especially beneficial, as discussed below, as a means for greatly increasing blood flow in the skin and penetration of the topical cream into the skin.

In preferred embodiments, the heating apparatus may be adjustable to a plurality of control settings, each control setting featuring a different amount of temperature fluctuation up and down within a preferred range for that control setting. Preferably, low, medium, and high control settings are used, which are characterized by low, medium and high average temperatures and low, medium, and high fluctuations in temperature, and these features and desired temperature waveforms may be achieved via various electronic and/electric control systems. The average temperature of the preferred control settings increases from low to medium to high, and most preferably, the amplitude of temperature fluctuation also increases, so that heat may be applied to the injured area at low temperatures for long periods of time or at high temperatures for short periods alternating with short periods of passive or active relative cooling so that the heating apparatus surface and the skin temperature drop relative to the peak condition. By “relative cooling” is meant dropping in temperature relative to the peak temperature, but not necessarily that the heating pad surface actually drops below the skin temperature to actively cool the skin.

At the preferred low setting, pulsing of the heating elements is controlled within a tight range without a significant risk of burning skin or body tissues. However, at the high setting, the heating elements attain significantly higher temperatures and attain significant variation between the highest (peak) temperature and the lowest (trough) temperature, in a special waveform that is adapted to optimize topical cream penetration without significant pain and burn risk. For example, in apparatus adapted for three or more control settings, the highest temperature setting preferably comprises heating element peak-to-trough differences of generally between about 15-20 degrees Fahrenheit. Between the low and high settings is the medium setting, which is between the two extremes in average temperature and in amplitude of temperature waveform.

When the heating pad is applied to the skin surface over an injured area, the large peak-to-trough temperature fluctuations produce periods of intense heat followed by periods of relative cooling of the surface of the heat pad and the skin, that is, relative to their temperature at the peak temperature condition. This cooling may be accomplished by passive cooling, wherein the heating pad surface and the skin lose heat relative to the peak condition due to heat loss into the surroundings and into the body. In such embodiments, the heating pad surface temperature remains above the skin temperature, but, due to reduced power, shut-off power, or other power modification, the heating pad surface temperature is dropping and the driving force to heat the skin is thereby reduced. Alternatively, but less preferably due to the expense of building such an embodiment, a heating device may be designed that actually switches from an active heating mode to an active cooling mode, by transitioning from periods wherein the apparatus surface is hotter than the skin to periods wherein the apparatus surface is cooler than the skin.

In the preferred embodiments, the application of intense heat for short periods of time during the treatment cycle dramatically increases skin permeability and, correspondingly, increases the effectiveness of the preferred analgesic cream or other topical creams by allowing the various components of the cream to penetrate deeply into the affected area. Extended time at high temperature can burn the skin, but, by limiting the amount of time at high temperature to repeated, brief periods of time, followed by repeated periods of relative cooling, the skin is exposed to the benefits of the high temperature without being burned. While the skin temperature cycles up and down in response to the heating pad temperature, it does not reach dangerous or painful temperatures. Consequently, the treatment according to the preferred embodiment, at which high heating pad element temperatures is achieved, is substantially improved without significantly increasing the risk of burning or discomfort.

In the preferred embodiment for pain treatment, the analgesic cream is placed between the heating pad and the injured area of the body so that the cream is effectively absorbed by the injured area. Unlike many conventional analgesics, the cream of the present invention is compatible with the heating pad treatment method. Preferably, the cream contains, at least, an analgesic component and anti-inflammatory components. More preferably, the cream contains specific amounts of analgesic and anti-inflammatory agents in addition to skin conditioners, emollients, herbal extracts and other natural components. These components, or the specific combination of these ingredients, may be said to be heat-enhanced such that upon heating, the cream penetrates deeply into the skin and injured area to reduce pain and discomfort.

### BRIEF DESCRIPTION OF THE DRAWINGS

FIG. 1 is a side view of one embodiment of the invention, in a kit including an embodiment of the invented heating pad and an embodiment of the invented treatment cream.

FIG. 2A is a side, perspective view of the heating pad, sleeve, foam sheet, and cream of the embodiment of FIG. 1.

FIG. 2B is a side, perspective view of the heating pad straps of the embodiment depicted in FIG. 1.

FIG. 3 is a side view of the embodiment of FIG. 2A in use on a body area in need of treatment, with the heating pad, foam layer, and sleeve shown in cross-section.

FIG. 4 is a graph of temperatures resulting from an example of operating a conventional heating pad with conventional temperature control.

FIG. 5 is a schematic graph of one embodiment of the invented heating pad element temperatures used for treatment according to an embodiment of the invented method.

FIG. 6 is a graph of the preferred heating pad temperature ranges and waveforms for one embodiment of a pain treatment method according to the present invention as measured in a variety of locations on the pad.

FIG. 7. is a graph of the control temperatures, wire trace temperatures, and skin temperatures when a low power treatment according to one embodiment of the invention is performed on the kidney area of a patient and on the spinal area of the patient.

FIG. 8 is a graph of the control temperatures, wire trace temperatures, and skin temperatures when a medium power treatment according to one embodiment of the invention is performed on the kidney area of a patient and on the spinal area of the patient.

FIG. 9 is a graph of the control temperatures, wire trace temperatures, and skin temperatures when a high power treatment according to one embodiment of the invention is performed on the kidney area of a patient and on the spinal area of the patient.

FIG. 10 is a front view of one embodiment of a facial heating mask that may be used for pain treatment or skin care according to methods of the invention.

FIG. 11 is a top view of one embodiment of heating mittens that may be used for pain treatment or skin care according to methods of the invention.

### DETAILED DESCRIPTION OF THE INVENTION

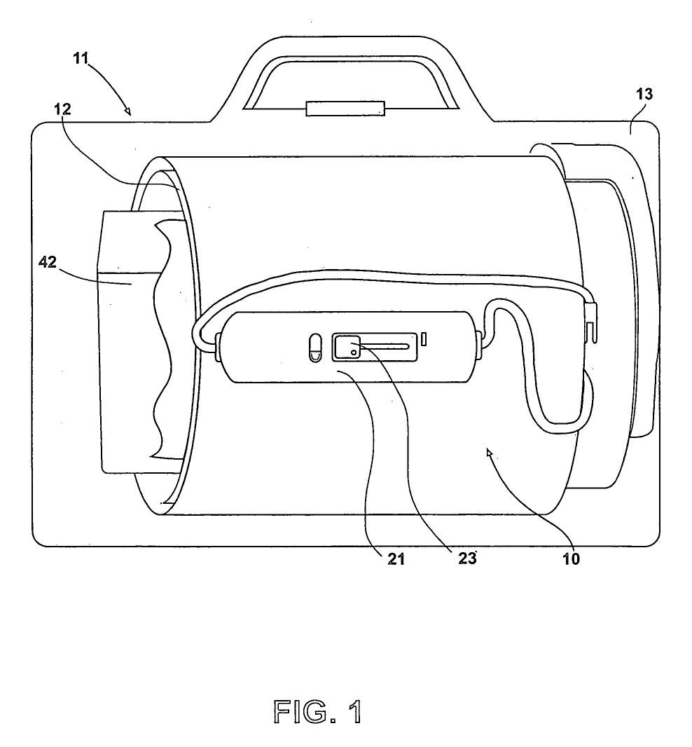

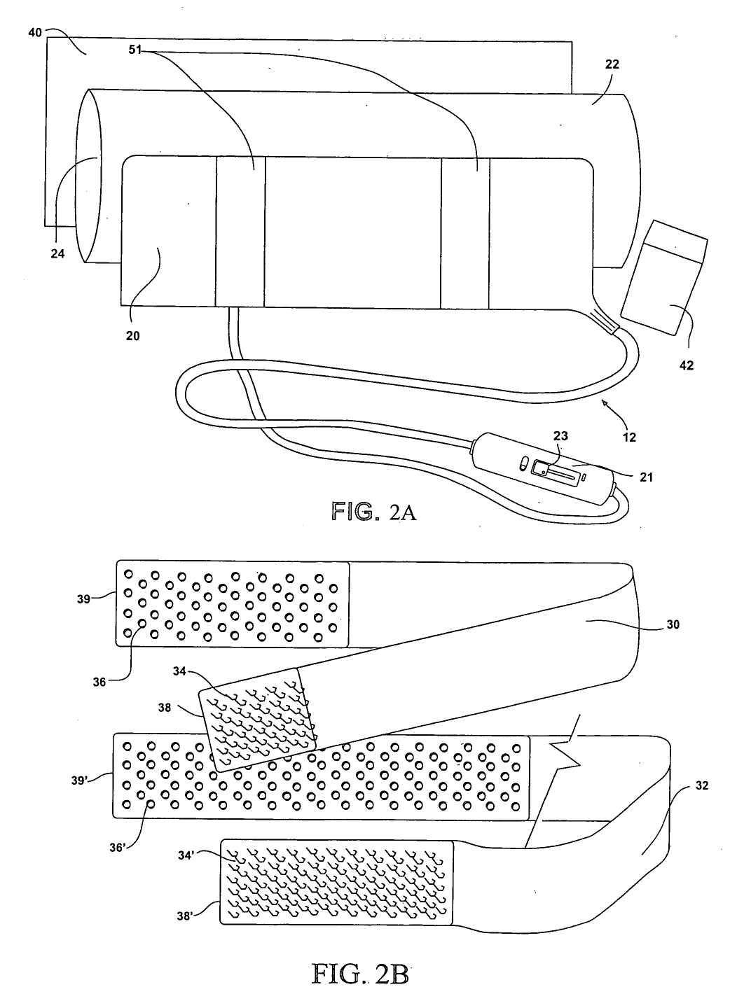

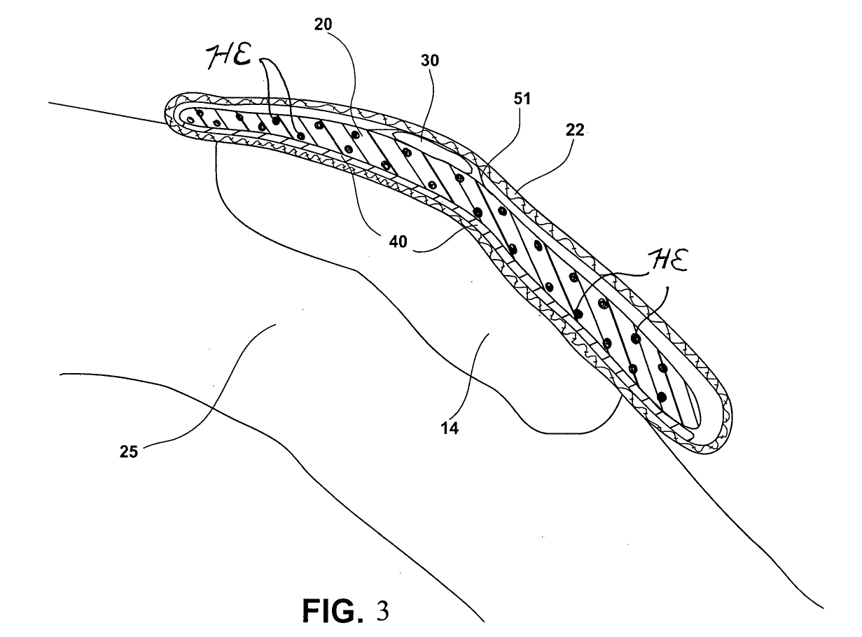

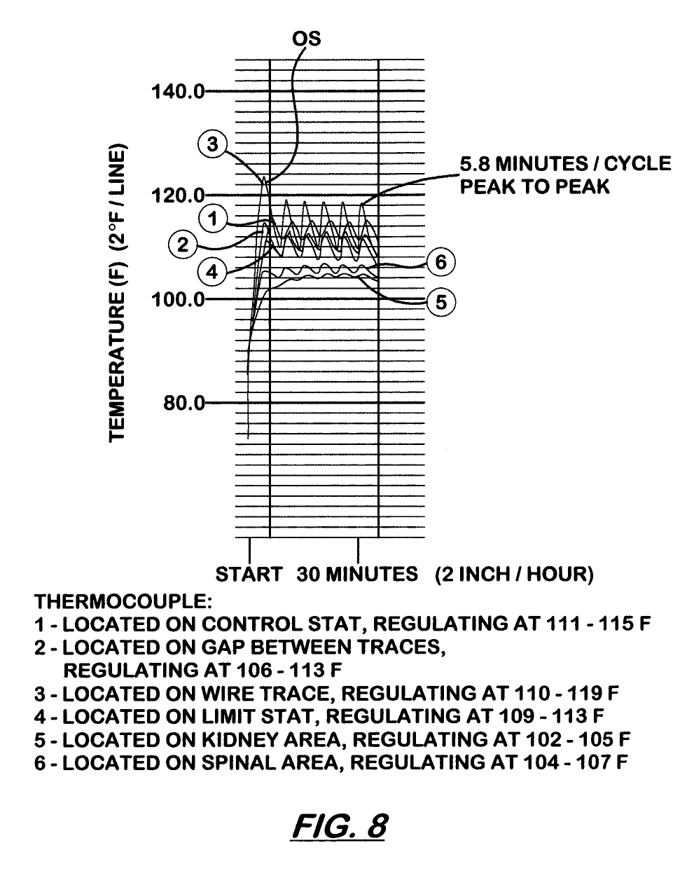

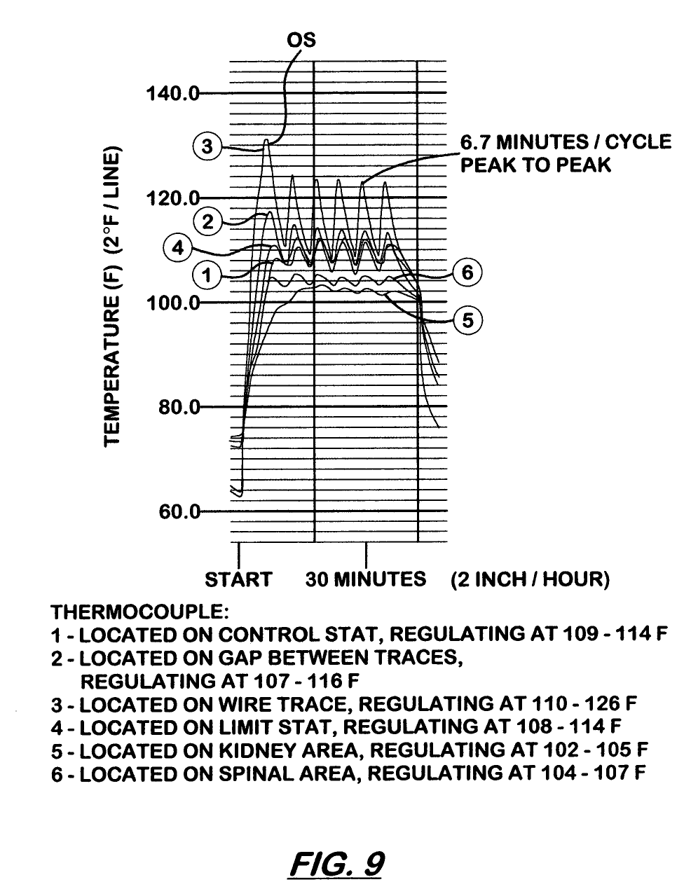

Referring now to FIGS. 1-3 and 5-11, there are shown some, but not the only, embodiments of the invented body tissue and skin treatment method and apparatus, which may include pulsed heating of body tissue and skin with various topical creams, including analgesic creams and/or skin moisturizing creams, for example. In this description and the claims, the term “topical cream” includes creams, ointments, lotions, and other substances in liquid and even solid form that may be applied to the skin for soothing, pain reduction, moisturizing, medicinal, herbal, and other treatment purposes.

The preferred method is an improved pain treatment system utilizing a thermostatically-controlled heating pad in conjunction with an analgesic cream to substantially increase penetration of the cream into the skin and body tissues. Controlled heating of the skin over an injured area relaxes musculature around an injured area, increases local blood flow to the area and substantially increases skin permeability. Consequently, the application of controlled, non-burning heat to an area with the invented heating pad system enhances the effectiveness of drug delivery to injured tissues via the topical analgesic.

In the preferred embodiment, the invented pain treatment system 10 comprises a thermostatically-controlled heating pad 12 and an analgesic topical cream 14. Other equipment may be supplied as part of the system in some embodiments. For example, in one kit 11 embodiment, a carrying case 13, such as the one shown in FIG. 1, may be provided to conveniently store and/or transport the individual system components. In another embodiment, a flexible sleeve 22 or a plurality of flexible straps 30, 32 may be provided with the heating pad and cream to secure the heating pad over the injured area when in use. The straps 30, 32 may comprise hook and loop portions (36, 36′, 34, 34′ on ends 39, 39′, 38, 38′). Or, the heating pad 20 itself may be adapted to have integral straps or sleeve members 51 through which an arm or leg may slide for attachment of the pad 20 to the patient. In yet another embodiment, a thin sheet of spongy foam 40 material may be supplied with the heating wrap and cream. The foam material may be moistened and inserted, preferably between the heating pad and cream, to generate moist heat above the injured area. These articles and/or other equipment may be supplied with the preferred heating pad and analgesic cream as needed to enhance the utility or convenience of the invented pain treatment system.

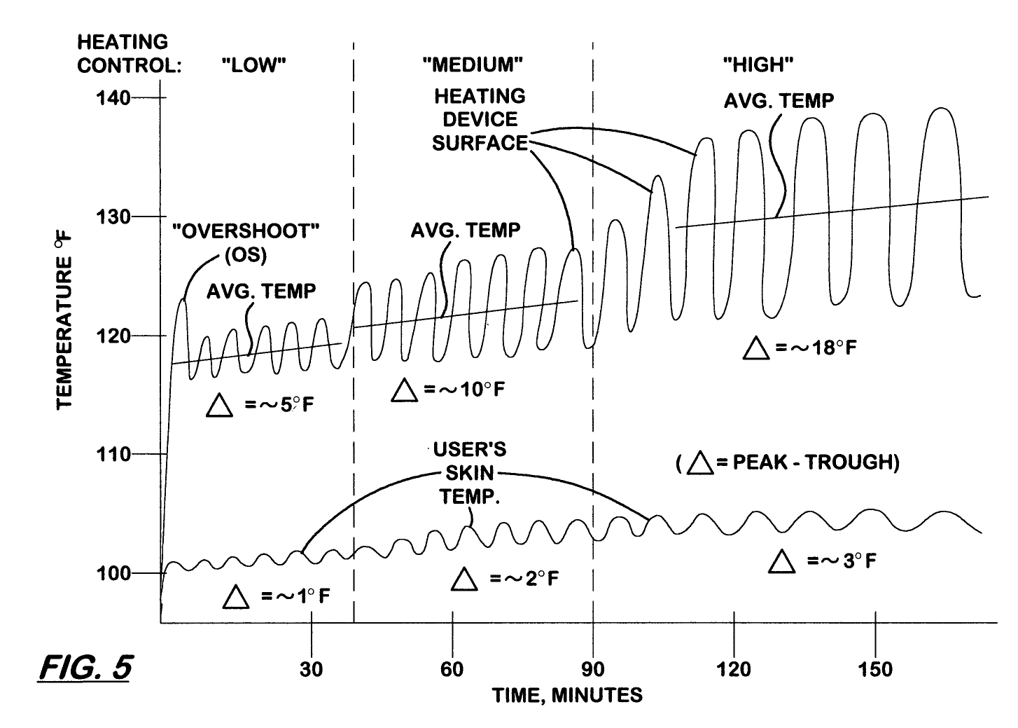

As shown in FIGS. 5-9, the heating pad of the preferred embodiment comprises specially-controlled heating elements (HE) contained within an otherwise conventional heating pad, wherein the preferred heating pad control (shown as control unit 21 and control switch 23 in FIG. 1) is adapted by electric/electronic configuration and/or programming to control temperature in a wave pattern of substantially larger temperature fluctuations than result from conventional heating pad control.

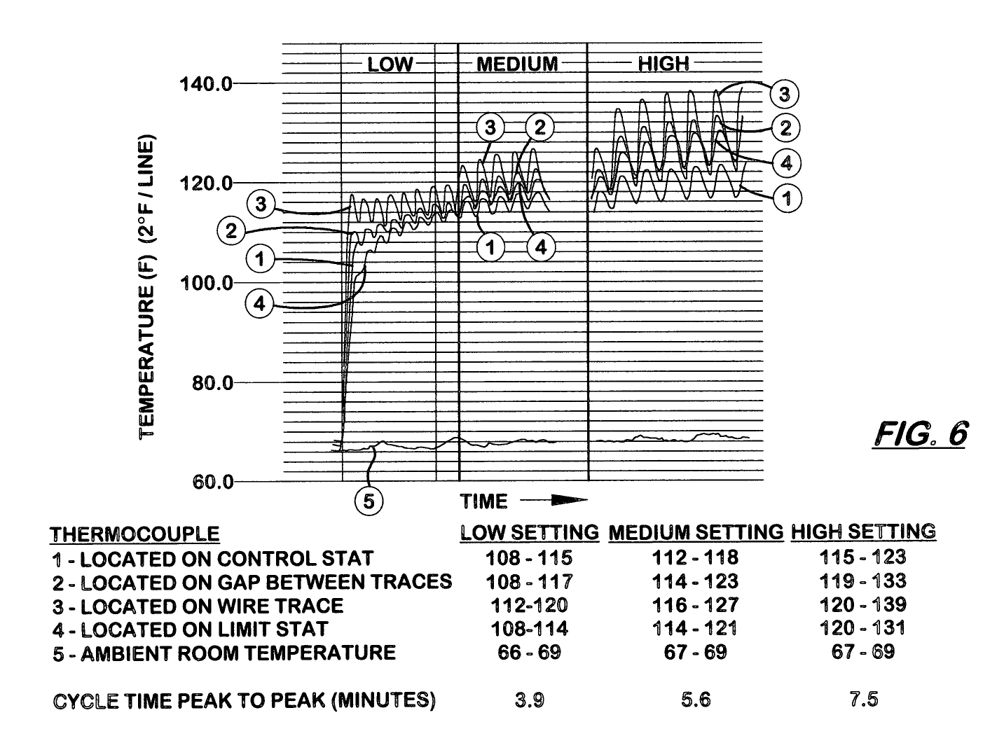

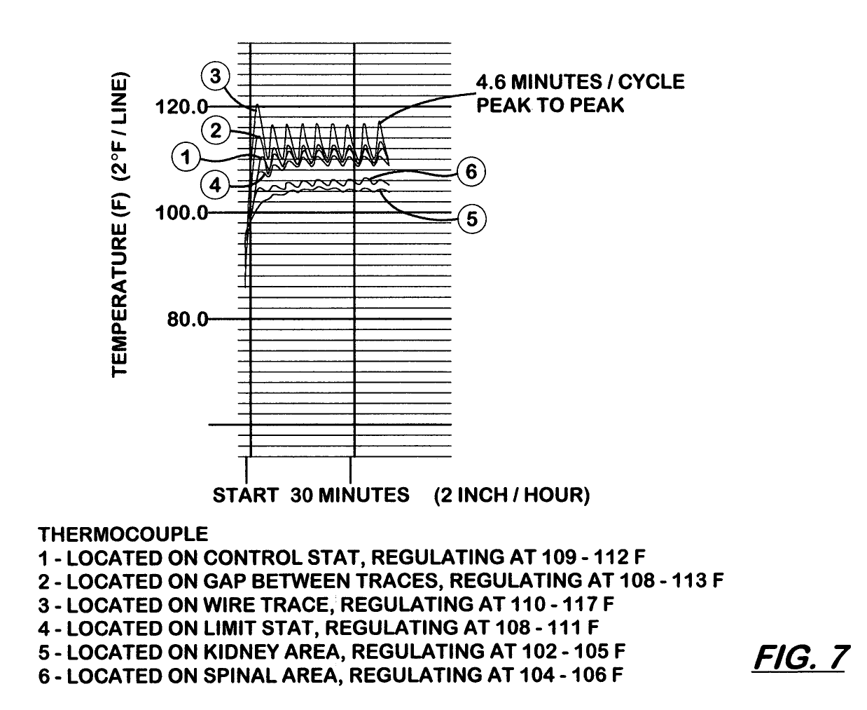

FIG. 5 illustrates schematically one embodiment of the invented apparatus and methods, wherein a heating pad exhibits three control settings, of low, medium, and high, and reaches large amplitude waveforms in the high control settings. FIG. 6 illustrates actual data from a “blanket test” of an embodiment of the invented heating pad, wherein a blanket test is a standard test for heating pads in which the pad is covered on both sides with a blanket. FIGS. 7-9 are actual data from an embodiment of the invented heating pad, showing skin temperature data in addition to other pad-related temperature measurements.

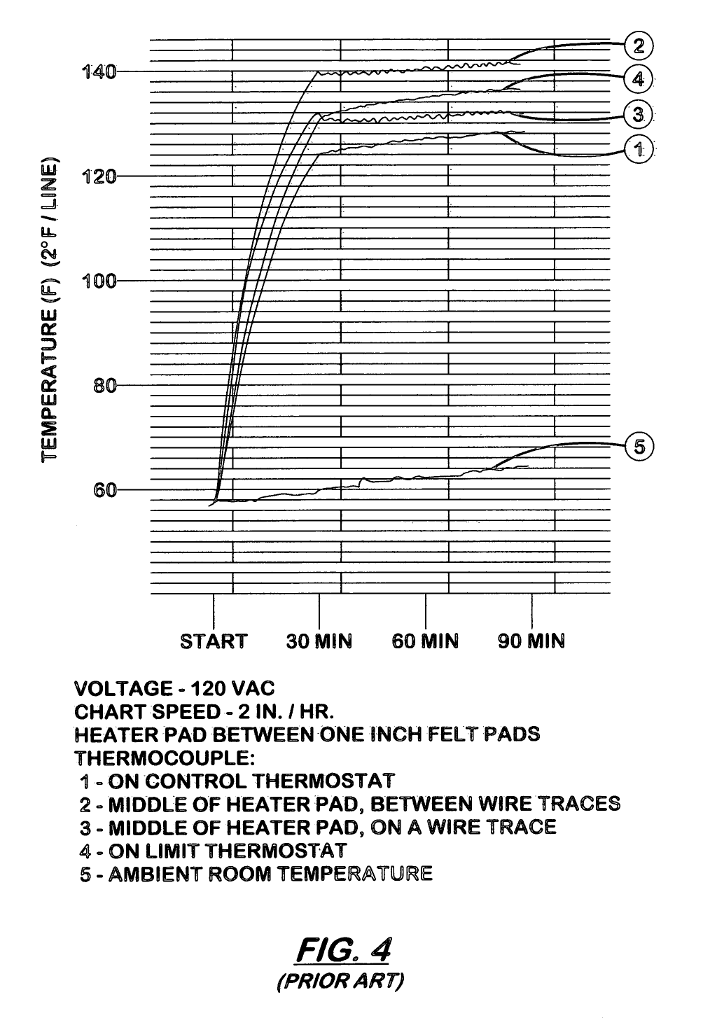

An example of conventional temperature control for a conventional heating pad is shown in FIG. 4. In FIG. 4, after a typical overshoot to the “overshoot temperature” (OS) at the beginning of heating, the temperature “lines-out” at the set point temperature, with very minimal (about 1 degree F.) fluctuation about the set point. As will be understood by those of skill in control, some minor fluctuation about the set point is normal, but conventional control allows for this fluctuation purposely to be very small. While a conventional heating pad controller controls a heating pad within 1-3 degrees Fahrenheit of each of its one to five separate temperature set-points, the preferred controller is adapted to establish temperature waveforms having at least 5 degrees difference, and more preferably 5-20 degrees difference, from trough to peak. Thus, in the preferred embodiment, periodic fluctuation of the heating element temperature between the minimum and maximum temperature prescribed for the particular device temperature setting is acceptable and, in fact, desirable. Once one skilled in the electronic/electric arts, and particularly in the control arts, reads and views this specification and the drawings, he/she will be able to build or program an appropriate controller according to the invention. The electronic/electric control for achieving the desired temperature waveforms, may include, for example, adjustment of the duration of intense heating and duration of relative cooling by turning on and off electricity to the heating elements at a single power level, respectively; power level adjustment or modification; active heating followed by active cooling; and other control methods that will be apparent to those skilled in the art. Therefore, it may be understood that the heating device may be controlled in waveforms by turning on power to heating elements to attain the peak and turning off power to let the heating elements attain the trough (by falling to the trough temperature) and/or by keeping the power on all the time but adjusting power downward to allow the temperature to fall to the trough, or other methods apparent to those of skill in the art.

Also, in the preferred embodiment, it is preferable and desirable to program/build the heating element(s) controller to produce heat in a fluctuating temperature range for several specified control settings, for example, a LOW, a MEDIUM, and a HIGH setting, as may best be seen in FIG. 5. The LOW setting is used for patients with sensitive skin or who are starting such treatment for the first time. The MEDIUM and HIGH settings are used for patients with more heat tolerance or who have adapted to the heat during previous periods of treatment with the invented apparatus.

For example, at the LOW temperature setting, the heating elements may reach, and fluctuate between, temperatures of generally about 112-120 degrees Fahrenheit, as measured on the surface of the heating pad (typically, the plastic surface, not the surface of any cloth covering) at the location of the heating wire/element. For example, at a MEDIUM temperature setting, the heating elements may achieve, and fluctuate between, temperatures of generally about 116 and 127 degrees F. For example, at the HIGH setting(s) the temperature of the heating elements preferably achieve and alternate significantly, and periodically, between about 120 degrees and 139 degrees Fahrenheit. The inventors envision that even higher peak temperatures in the range of about 140-150 degrees F. may also be beneficial. In the preferred embodiment, a periodic fluctuation, or pulsing, of the heating elements between the peak and trough at the upper and lower extremes of these approximate ranges is important to the specific function of the heating pad, especially at the highest settings. Preferably, the temperature waveforms in each control setting repeat approximately every 2-12 minutes, and more preferably 3-8 minutes, and preferably, a treatment comprises exposure of the skin to at least 3 cycles (at least 3 temperature waves from trough to peak) at each control setting, or to at least 6 cycles at a single control setting, for example, if only one control setting is used during a treatment session. The trough temperatures may be provided by shutting off or reducing heat, or, in alternative embodiments, by actually actively cooling the pad and/or skin by a cooling means such as a cooling liquid or other temperature reduction.

At the lower temperature setting(s), fluctuation, or pulsing, of the heating pad temperature is preferably restricted within a tight range because the particular body tissues/patients to which the heat treatment is applied are typically adapted/accustomed to tolerate low temperature heating for extended periods of time. For some individuals, an effective increase in skin surface temperature and effective analgesic or other drug penetration may be accomplished via long periods of heating at the lower pad setting(s). However, at the higher temperature setting(s), the pulsing of the heating elements between a relatively high maximum and low minimum value is integral to the especially-preferred embodiments because the fluctuation enables the application of intense heat to the skin for controlled periods of time followed by sufficient periods of skin cooling to prevent burning. The application of intense heat to the skin in this manner relaxes musculature surrounding an injured area, increases blood flow in and to the area and increases skin permeability which promotes penetration of the analgesic cream into the skin and body tissues. While the skin temperature cycles up and down in response to the heating apparatus temperature, as illustrated in FIGS. 5, 7-9, the skin temperature is moderated by the relative cooling periods and by the increases blood flow that carries heat away from the skin surface, and so the skin does not reach temperatures that may burn the skin. As a result, the specific heating and cooling cycle can be carried out for a period of time to enhance pain treatment without substantially increasing the risk of burning or discomfort.

Normal skin surface temperature is generally around 91 or 92 degrees Fahrenheit. According to the preferred embodiment of the invented pain treatment system (particularly the specific heating and cooling cycle encouraged by the heating pad) skin surface temperature is increased to generally within the range of 98-106 degrees Fahrenheit. Typically, at least a 5 degree increase in skin surface temperature is required to noticeably increase skin permeability. At 98° F., skin permeability is approximately doubled. Permeability and blood flow continue to increase above this temperature. However, at temperatures generally above about 106° F., burning of the skin may occur. Therefore, in general, increase in skin surface temperature promotes deeper penetration of the analgesic cream into the skin and body tissues but there is limit to what a person can tolerate without heat-induced pain or burning. Further however, as described below, skin temperature is believed by the inventors not to be the only indicator and not the only factor in accomplishing increased skin permeability and blood flow.

As illustrated by FIGS. 7-9, the three preferred heating levels labeled as the LOW setting, MEDIUM setting, and the HIGH setting, corresponding to low, medium, and high heat input, respectively, and result in elevated kidney area skin and spinal area skin temperatures. For all three heat inputs in FIGS. 7-9, however, the kidney area skin stays within a range of about 104-105 degrees F. substantially all through the treatment. For all three heat inputs in FIG. 7-9, the spinal area skin stays within a range of about 105-106 degrees F. substantially all through the treatment. This difference between the kidney area and the spinal area may be attributed to the relative amounts of blood vessels in the two skin areas.

It may be surprising that the skin temperatures do not change to a greater extent when the heat input is so greatly increased by moving from the LOW setting, to the MEDIUM setting and finally to the HIGH setting, but this is explained by the inventors as the increased blood flow response to the heat input. As the greatly increased blood flow carries heat away from the skin and deeper into the body, skin temperature is moderated. Also, the inventors believe that sweat glands tend to open at or near the intense heat input condition, further helping to moderate skin temperature. Thus, the analgesic or other cream penetration is believed to be greater during the periods of high heat input than may be indicated by the measured skin temperature increase. Therefore, this greatly increased penetration may be accomplished via short periods of heating at the higher pad setting(s) wherein the brief periods of high peak temperature are especially effective without creating a burn or pain condition. The inventors believe that the brief high temperature exposure, by high power input into the heating pad and therefore high heat input into the skin, followed by periods of relative cooling (for example, by turning off of the heating elements) is especially effective because the high temperature dilates the blood vessels due to relaxation of involuntary muscle contained in their walls, thus increasing blood flow to the skin. The permeability of the skin is increased, allowing better penetration of the topical cream. The heat to the skin reflexly produces effects in the deeper portion of the body. It induces muscle relaxation, increases blood supply, and stimulates metabolic activity. Relaxation of muscular tissue results in relief of pain, which may be due to rigidity and spasm in tissues. Local hot applications have some reflex effect on deep organs. The relative cooling period, after the high temperature exposure, allows the skin to avoid damage and avoid burn while withstanding a higher peak temperature.

The invention teaches the benefits, therefore, of using embodiments of a heating device that pulse at a high temperature and with large amplitude temperature waveforms. Multiple settings, each having a different absolute temperature level and different amplitude of pulsing/fluctuation are preferred, however, to give the user various options for his/her treatment. The lower device settings may be applied in the initial phases of treatment to prepare the patient for higher temperature settings. In many patients, heat sensitivity decreases as the low temperature heat treatment progresses, so such patients may start with low heat input treatment and then progress in the same treatment session or in a later session to higher heat input. Optionally, the invention may include apparatus capable of only one or more of such settings.

The analgesic of the preferred embodiment comprises a topical cream composed of substantially heat-enhanced ingredients that are intended to reduce or eliminate discomfort and pain in an injured area. The components of the cream may be described as heat-enhanced or heat-activated to the extent that they are not heat-producing or irritating and become more fluid or more aromatic at high temperatures so that the likelihood of safely penetrating the skin and body tissues is sufficiently increased. Preferably, the topical analgesic cream comprises at least analgesic and anti-inflammatory agents. For example, the cream may contain the analgesic and anti-inflammatory substances methyl salicylate and aloe, respectively. Other analgesics such as menthol may also be used and alternative, or additional, anti-inflammatory agents are likely to be foreseen by one skilled in the art. More preferably, the cream contains at least a salicylate component, anti-inflammatory agents and a linoleic ester. An especially preferred linoleic ester is derived from zwitterionic-fatty-acid (ZFA). In a further preferred embodiment, the cream contains analgesic and anti-inflammatory agents in addition to the ZFA, emollients, skin conditioners, herbal extracts and other natural components. The topical cream components may be selected from the listed group comprising: methyl salicylate, menthol, linoleic ester, aloe, balsam leaf extract, willow extract, horse-chestnut extract, coconut waxes, allantoin, panthenol, jasmine essence, Echinacea, basabalol and sodium benzoate. Preferably, the analgesic (salicylate) component comprises generally about 10-20 weight percent, linoleic ester comprises about 1-10 weight percent and the anti-inflammatory agents (aloe) comprise about 40-60 weight percent, and most preferably about 50 weight percent, of the mixture. The remaining 10-49 wt-% of the topical cream is preferably selected from the above listed group, wherein the amount of each added ingredient selected from the above listed group generally does not exceed 5 weight percent of the total topical cream. The selections from the above listed group and the amounts of the selected components may be determined according to the formulator's preferences, to arrive at a topical cream according to their preferred customer's wishes or their preferred blending techniques. Also, the inventors' envision that other components besides those in the above listed group may be added, for example, with the described topical cream being mixed with “other ingredients” so that the described topical cream is, for example, approximately 95% wt of the final substance and the “other ingredients” are approximately 5% of the final substance, or, for example, the described topical cream being approximately 90 wt % of the final substance and the other ingredients being approximately 10 wt % of the final substance.

In the preferred analgesic, capsaicin or capsicum-derived substances and skin irritants are excluded from the topical cream composition. In conventional analgesic creams, capsaicin and capsicum-derived ingredients are present up to about 0.25 weight percent to promote an exothermic reaction that heats the skin. According to the present invention, this reaction is unnecessary and undesirable. Consequently, the presence of such ingredients in the topical analgesic of the present invention is minimized or eliminated. In the preferred analgesic cream, capsaicin and capsicum-derived substances are not present in amounts above about 0.025 weight percent. More preferably, these ingredients are limited to below 50 parts-per-million.

According to the preferred embodiment of the present invention, the analgesic cream is applied to the skin surface to begin the pain treatment process. The analgesic is spread over the skin such that the injured area is sufficiently coated with the topical cream. The heating pad is then secured over the injured area using the sleeve, straps or other means and activated. The desired heating pad setting is then selected and the heating and cooling cycle begins. As the pain treatment cycle unfolds, the skin is subjected to controlled, non-burning heat to encourage deep penetration of the topical cream into the skin and/or injured body tissues. As the cream ingredients contact the body tissues, pain and discomfort are reduced. The treatment cycle may continue as time permits or until pain and discomfort are substantially eliminated. Preferably, treatment begins at a low temperature setting and progresses to the higher settings as the patient becomes less sensitive to, and better able to tolerate, higher temperatures. The following example outlines one of many possible progressions of pain treatment according to the present invention:

### EXAMPLE

Step 1. Apply a sufficient quantity of the invented analgesic or similar cream to the skin over an injured area of the body.

Step 2. Apply a heating device to the injured area on which has been placed the topical cream.

Step 3. Activate the heating device and set the device to its lowest temperature setting (i.e. LOW). At this setting, the temperature of the device heating elements preferably cycles between peaks of generally 118-120° F. and troughs of about 112-114° F. Each cycle (from one peak to the next) preferably requires approximately 4 minutes.

Step 4. After treatment at the lowest device temperature setting, as outlined above for approximately 20-45 minutes, adjust the device to a higher setting (i.e. MEDIUM). At this setting, the temperature of the heating elements preferably cycles between peaks of generally 124-127° F. and troughs of about 116-118° F. in the similar period as is used for the LOW setting, for example, cycles of approximately 4-6 minutes.

Step 5. After treatment at the medium device temperature setting, as outlined above, for approximately 20-45 minutes, adjust the device to a higher setting (HIGH). At this setting, the temperature of the heating elements preferably cycles between peaks of generally 135-138° F. and troughs of about 120-124° F., with the cycles being approximately 6-8 minutes long. Pain treatment according to this HIGH heating step may continue until the highest temperature setting is continued for approximately 20-45 minutes, or until it becomes intolerable or until pain is effectively eliminated.

Step 6 End Treatment and remove the heating pad.

FIGS. 5 and 6 illustrates the above steps 1-5, but other temperatures may be found to be effective. It may be noted from FIGS. 5-7 that the LOW temperature treatment may comprise exposure of the skin to heating by temperature wave of about 5-10 degrees F. from peak to trough. Alternatively, the LOW control setting may comprise controlling to substantially a constant temperature, for example, a conventional control system for controlling to within 2-3 degrees F. of the set-point. FIGS. 5, 6 and 8 illustrate that the MEDIUM temperature treatment may comprise exposure of the skin to heating by temperature waves of about 10-15 degrees F. from peak to trough. FIGS. 5, 6, and 9 illustrate that the HIGH temperature treatment may comprise exposure of the skin to heating by temperature waves of about 15-20 degrees F. from peak to trough. While these waveforms are preferred, and the period of each waveform is preferred to be about 3-8 minutes, other temperatures, amplitudes, and periods may be used. For example, a heating apparatus and control system may include only two control settings, for example, a low control setting or moderate or little temperature fluctuation, and a high control setting with peak-trough differences in the range of about 10-20 degrees F. Or, for patients who are less sensitive or for whom caretakers will be present, a heating device with a single control setting in the high range may be effective. Particularly, a heating apparatus having at least one control setting with a waveform of at least 10 degrees F. difference between peak and trough is preferred.

Therefore, as alternatives to the above treatment example, a user may select only the LOW control setting for about ½ hour on Day 1, followed by about ½ hour on control setting MEDIUM for Day 2 and about ½ hour on control setting HIGH for Day 3. Or, a veteran user of the heating apparatus and method, may go directly to HIGH for the entire treatment and all days/sessions of treatment.

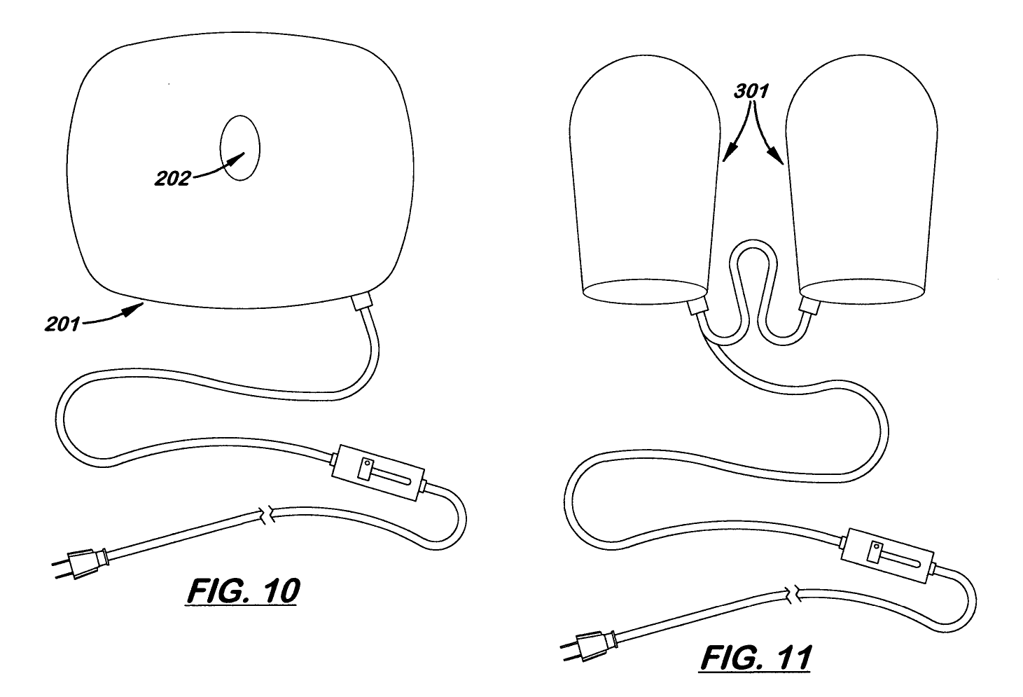

As illustrated in FIGS. 1, 2A, 3, 10 and 11, many shapes may be used for the heating device according to the invention. Rectangular, round, sleeve-shaped, or other shapes may be used as desired for various parts of the body. FIG. 10 illustrates an embodiment particularly adapted for facial skin treatment, that is, a face mask 201 with a nose hole or aperture 202 for allowing breathing. FIG. 11 illustrates mittens 301 that are well-adapted for treatment of the hands, for example, for arthritis or other joint ailments, or for hand skin moisturizing, for example. While embodiments of the preferred temperature control have been described, the remainder of the heating pad may typically be made according to conventional methods, for example, heating elements inside appropriate padding or webbing to hold the elements in place, covered by a plastic cover, with or without an outer cloth casing for improved aesthetics and comfort.

The Figures and the above description illustrate that a preferred means for measuring and/or controlling temperature is to measure the temperature waveform at the wire trace, and this corresponds to a temperature measurement on the outside of the heating pad (typically on the surface of the plastic casing of the pad) at the location on the outside of the pad that is nearest the wire (or other heating elements in the pad). Other temperatures may be used for control as will be understood by those of skill in the art. Therefore, the term “temperature of the heating device” in the claims is meant the temperature of the outer surface of the heating device on average that is available for placement against the user's skin, and temperature of the heating device at or near the heating element” is meant the temperature of the outer surface of the heating device within ½ inch of the heating element.

While the preferred cream/drug treatment is an analgesic topical cream made as described above, other substances such as other creams, ointments, lotions, and medicines, drugs, and treatments may also be used with the invented heating pad and control system. For example, another preferred topical cream may be of the type commonly referred to as “skin care creams” and may include moisturizing agent(s) that may be selected by one of skill in the skin care art. Further, other heating devices besides a heating pad may be used, preferably but not necessarily with the preferred pulsed heating features. Further, the invention in some embodiments comprises the invented analgesic cream with non-pulsing heating devices and other heating temperatures and control systems besides the invented pulsed waveform heating system.

Although this invention has been described above with reference to specific means, materials and embodiments, it is to be understood that the invention is not limited to these disclosed particulars, but extends instead to all embodiments within the scope of the following claims.

I claim:

1. A body tissue treatment system comprising:
a variable-heat heating device having a control setting, wherein said setting is adapted to control temperature of the heating device to rise to a peak temperature and to fall to a trough temperature in a waveform so that there is at least 5 degrees F. difference between the peak temperature and the trough temperature.

2. A body tissue treatment system as in claim 1, wherein said setting is adapted to control temperature of the heating device in a waveform having a difference between the peak temperature and the trough temperature of 10-20 degrees.

3. A body tissue treatment system as in claim 1, comprising a plurality of control settings adapted to control temperature of the heating device in a waveform having differences between the peak temperature and the trough temperature of at least 5 degrees F.

4. A body tissue treatment system as in claim 1, comprising at least two control settings, each adapted to control temperature of the heating device in a waveform having a difference between the peak temperature and the trough temperature of at least 5 degrees F., and wherein the difference between peak and trough of the temperature waveform of one of said two control settings is greater than the difference between the peak temperature and the trough temperature of the other of said two control settings.

5. A body tissue treatment system as in claim 1, wherein the heating device has a low control setting, a medium control setting, and a high control setting, and wherein said low control setting exhibits a temperature fluctuation waveform having a difference between the peak and trough temperatures of 5-10 degrees F.

6. A body tissue treatment system as in claim 5, wherein said medium control setting exhibits a temperature fluctuation waveform having a difference between the peak and trough temperatures of 10-15 degrees F.

7. A body tissue treatment system as in claim 5, wherein said high control setting exhibits a temperature fluctuation waveform having a difference between the peak and trough temperatures of 15-20 degrees F.

8. A body tissue treatment system as in claim 1, further comprising a topical cream for placement between the heating device and a user's skin.

9. A body tissue treatment system as in claim 8, wherein the topical cream is an analgesic cream for treatment of pain.

10. A body tissue treatment system as in claim 8, wherein the topical cream is an analgesic cream comprising methyl salicylate.

11. A body tissue treatment system as in claim 8, wherein the topical cream is an analgesic cream comprising zwitterionic-fatty-acid.

12. A body tissue treatment system as in claim 10, wherein the topical cream is an analgesic cream comprising zwitterionic-fatty-acid.

13. A body tissue treatment system as in claim 8, wherein the topical cream is a skin care cream comprising a skin moisturizing agent.

14. A body tissue treatment system comprising:
a heating device having heating elements and a controller for controlling the temperature on an outer surface of the heating device;
said controller being adapted to control the heating elements in a temperature waveform oscillating between a peak temperature and a trough temperature, so that there is a difference between the peak temperature and the trough temperature in the range of at least 5 degrees and so that the heating device raises a user's skin surface temperature at least 5 degrees F.; and
the treatment system further comprising:
a topical cream for placement between said outer surface of the heating device and a user's skin, wherein said peak temperature increases blood flow in, and permeability of, the user's skin so that the topical cream penetrates deeper into the skin.

15. A body tissue treatment system as in claim 14, wherein the heating device has a plurality of control settings, and each of said control settings being adapted to create a temperature waveform having a different peak-to-trough temperature difference.

16. A body tissue treatment system as in claim 14, wherein the heating device has at least two control settings, and one of said two control settings is adapted to create a temperature waveform having a peak-to-trough temperature difference that is greater than the other of said two control settings.

17. A body tissue treatment system as in claim 15, wherein said plurality of control settings comprises a low temperature setting, a medium temperature setting, and a high temperature setting, wherein the low power setting exhibits a peak-to-trough temperature difference in the range of 5-10 degrees F.

18. A body tissue treatment system as in claim 17, wherein said the medium power setting has a peak-to-trough temperature difference in the range of 10-15 degrees F.

19. A body tissue treatment system as in claim 17, wherein said high power setting has a peak-to-trough temperature difference in the range of 15-20 degrees F.

20. A body tissue treatment system as in claim 14, wherein the topical cream is an analgesic cream for treatment of pain.

21. A body tissue treatment system as in claim 14, wherein the topical cream is an analgesic cream comprising methyl salicylate.

22. A body tissue treatment system as in claim 14, wherein the topical cream is an analgesic cream comprising zwitterionic-fatty-acid.

23. A body tissue treatment system as in claim 14, wherein the topical cream is a skin moisturizing cream.

24. A body tissue treatment system as in claim 14, wherein the topical cream is a skin moisturizing cream and the heating device is a face mask having a breathing hole for placement around the user's nose.

25. A body tissue treatment system as in claim 14, wherein the heating device is a pair of heated mittens for placement on the user'hands.
